(19) United States
(12) Patent Application Publication
(10) Pub. No.: US 2020/0085287 A1
(43) Pub. Date: Mar. 19, 2020

(54) MEDICAL IMAGING DEVICE AND ENDOSCOPE

(71) Applicant: Sony Corporation, Tokyo (JP)

(72) Inventors: Matthew LAWRENSON, Lausanne (CH); Naoyuki HIROTA, Stuttgart (DE)

(73) Assignee: Sony Corporation, Tokyo (JP)

(21) Appl. No.: 16/495,105

(22) PCT Filed: Feb. 22, 2018
(86) PCT No.: PCT/JP2018/006550

§ 371 Date: Sep. 18, 2019

(30) Foreign Application Priority Data
Mar. 29, 2017 (EP) 17163668.1

(51) Int. Cl. A61B 1/05; A61B 1/06; A61B 1/00

(52) U.S. Cl. CPC A61B 1/05; A61B 1/0638; A61B 1/00149; A61B 1/0684; A61B 1/0676; A61B 1/00006

(57) ABSTRACT

A medical imaging device comprising a Vertical Cavity Surface Emitting Laser configured to illuminate an object using one of a plurality of light conditions, an image sensor configured to capture an image of the object, and circuitry configured to: control the Vertical Cavity Surface Emitting Laser to illuminate the object using light having a first light condition; control the image sensor to capture a first image of the object with the light having the first light condition; control the Vertical Cavity Surface Emitting Laser to illuminate the object with light having a second, different, light condition; control the image sensor to capture a second image of the object illuminated with the light having the first light condition; and determine information of the object based on the first image and the second image.

### TECHNICAL FIELD

The present disclosure relates to a medical imaging device and endoscope.

### BACKGROUND ART

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present disclosure.

A problem when performing endoscopy (such as medical endoscopy or industrial endoscopy) or any kind of medical imaging is to identify the path of fluids through a system. In the example of medical endoscopy, there is a problem of identifying the veins, capillaries and arteries through which blood flows. This is especially the case where biopsies or other invasive procedures are required and major blood lines are to be avoided.

It is an aim of the present disclosure to address this issue.

Another problem with endoscopy is the similarity of surfaces in terms of colour and texture. This means that the arrangement of objects and surfaces being observed may confuse the user. This problem is particularly acute where specular reflections from a single source of illumination occur.

It is an aim of the present disclosure to address this issue.

### CITATION LIST

### Non Patent Literature

[NPL 1] ‘Gradient and Curvature from Photometric Stereo Including Local Confidence Estimation’, Robert J. Woodham, Journal of the Optical Society of America A (11)3050-3068, 1994.

[NPL 2] ‘Shape Reconstruction from Shadows and Reflections’, Silvio Savarese, PhD Thesis, California Institute of Technology, 2005.

### SUMMARY

According to embodiments, medical imaging device is provided that comprises a Vertical Cavity Surface Emitting Laser configured to illuminate an object using one of a plurality of light conditions, an image sensor configured to capture an image of the object, and circuitry configured to: control the Vertical Cavity Surface Emitting Laser to illuminate the object using light having a first light condition; control the image sensor to capture a first image of the object with the light having the first light condition; control the Vertical Cavity Surface Emitting Laser to illuminate the object with light having a second, different, light condition; control the image sensor to capture a second image of the object illuminated with the light having the first light condition; and determine information of the object based on the first image and the second image.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 is a view depicting an example of a schematic configuration of an endoscopic surgery system to which the technology according to an embodiment of the present disclosure can be applied.

FIG. 2 is a block diagram depicting an example of a functional configuration of the camera head and the CCU depicted in FIG. 1.

FIG. 3 schematically shows two particular embodiments describing the relationship between a lens arrangement and the light source apparatus in the endoscope system of FIG. 1.

FIG. 4 shows a graph 500 of the Molar extinction coefficient versus wavelength for deoxygenated haemoglobin and oxygenated haemoglobin.

FIG. 5 shows a flow chart explaining a process according to embodiments of the disclosure.

FIGS. 6A and 6B show a system according to embodiments of the disclosure.

FIGS. 6A and 6B show a system according to embodiments of the disclosure.

FIGS. 7 and 8 show a MEMs actuated mirror arrangement according to embodiments.

FIGS. 7 and 8 show a MEMs actuated mirror arrangement according to embodiments.

FIGS. 9, 10A and 10B show one MEMs actuated mirror arrangement in an endoscope according to FIG. 3.

FIGS. 9, 10A and 10B show one MEMs actuated mirror arrangement in an endoscope according to FIG. 3.

FIGS. 9, 10A and 10B show one MEMs actuated mirror arrangement in an endoscope according to FIG. 3.

FIG. 11 shows another MEMs actuated mirror arrangement in an endoscope system of FIG. 1.

FIG. 12 shows a system describing determining the topography of an object using embodiments of the disclosure.

FIG. 13 shows diagrams explaining the system of FIG. 12 in more detail.

FIG. 14 shows a flow chart explaining a process according to embodiments of the disclosure.

### DESCRIPTION OF EMBODIMENTS

Referring now to the drawings, wherein like reference numerals designate identical or corresponding parts throughout the several views.

Application

The technology according to an embodiment of the present disclosure can be applied to various products. For example, the technology according to an embodiment of the present disclosure may be applied to an endoscopic surgery system, surgical microscopy or medical imaging device or other kind of industrial endoscopy in, say pipe or tube laying or fault finding.

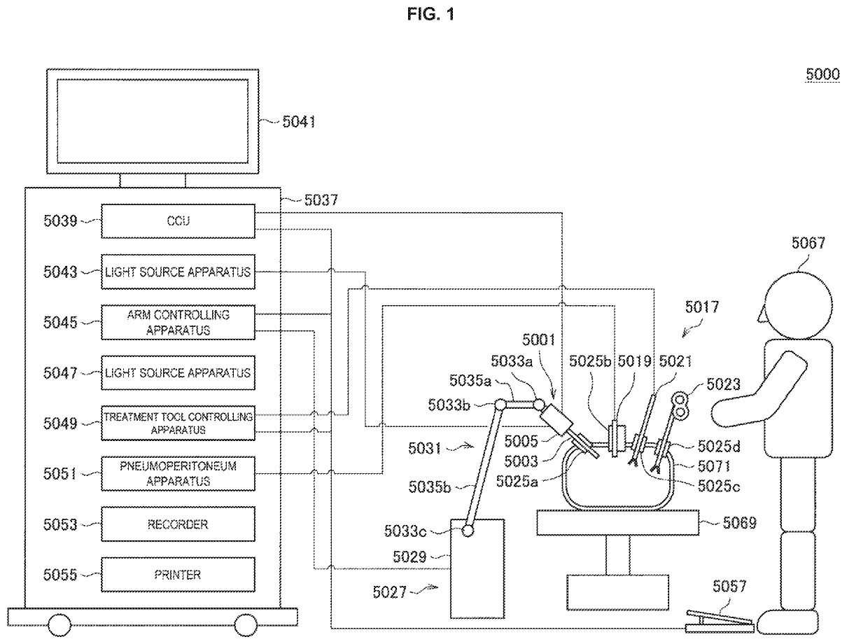

FIG. 1 is a view depicting an example of a schematic configuration of an endoscopic surgery system 5000 to which the technology according to an embodiment of the present disclosure can be applied. In FIG. 1, a state is illustrated in which a surgeon (medical doctor) 5067 is using the endoscopic surgery system 5000 to perform surgery for a patient 5071 on a patient bed 5069. As depicted, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d are used to puncture the abdominal wall. Then, a lens barrel 5003 of the endoscope 5001 and the other surgical tools 5017 are inserted into body lumens of the patient 5071 through the trocars 5025a to 5025d. In the example depicted, as the other surgical tools 5017, a pneumoperitoneum tube 5019, an energy treatment tool 5021 and forceps 5023 are inserted into body lumens of the patient 5071. Further, the energy treatment tool 5021 is a treatment tool for performing incision and peeling of a tissue, sealing of a blood vessel or the like by high frequency current or ultrasonic vibration. However, the surgical tools 5017 depicted are mere examples at all, and as the surgical tools 5017, various surgical tools which are generally used in endoscopic surgery such as, for example, a pair of tweezers or a retractor may be used.

An image of a surgical region in a body lumen of the patient 5071 imaged by the endoscope 5001 is displayed on a display apparatus 5041. The surgeon 5067 would use the energy treatment tool 5021 or the forceps 5023 while watching the image of the surgical region displayed on the display apparatus 5041 on the real time basis to perform such treatment as, for example, resection of an affected area. It is to be noted that, though not depicted, the pneumoperitoneum tube 5019, the energy treatment tool 5021 and the forceps 5023 are supported by the surgeon 5067, an assistant or the like during surgery.

(Supporting Arm Apparatus)

The supporting arm apparatus 5027 includes an arm unit 5031 extending from a base unit 5029. In the example depicted, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endoscope 5001 can be implemented.

(Endoscope)

The endoscope 5001 includes the lens barrel 5003 which has a region of a predetermined length from a distal end thereof to be inserted into a body lumen of the patient 5071, and a camera head 5005 connected to a proximal end of the lens barrel 5003. In the example depicted, the endoscope 5001 is depicted which includes as a hard mirror having the lens barrel 5003 of the hard type. However, the endoscope 5001 may otherwise be configured as a soft minor having the lens barrel 5003 of the soft type.

The lens barrel 5003 has, at a distal end thereof, an opening in which an objective lens is fitted. A light source apparatus 5043 is connected to the endoscope 5001 such that light generated by the light source apparatus 5043 is introduced to a distal end of the lens barrel by a light guide extending in the inside of the lens barrel 5003 and is irradiated toward an observation target in a body lumen of the patient 5071 through the objective lens. It is to be noted that the endoscope 5001 may be a direct view mirror or may be a perspective view mirror or a side view mirror.

An optical system and an image pickup element are provided in the inside of the camera head 5005 such that reflected light (observation light) from an observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 5039. It is to be noted that the camera head 5005 has a function incorporated therein for suitably driving the optical system of the camera head 5005 to adjust the magnification and the focal distance.

It is to be noted that, in order to establish compatibility with, for example, a stereoscopic vision (three dimensional (3D) display), a plurality of image pickup elements may be provided on the camera head 5005. In this case, a plurality of relay optical systems are provided in the inside of the lens barrel 5003 in order to guide observation light to each of the plurality of image pickup elements.

(Various Apparatus Incorporated in Cart)

The CCU 5039 includes a central processing unit (CPU), a graphics processing unit (GPU) or the like and integrally controls operation of the endoscope 5001 and the display apparatus 5041. In particular, the CCU 5039 performs, for an image signal received from the camera head 5005, various image processes for displaying an image based on the image signal such as, for example, a development process (demosaic process). The CCU 5039 provides the image signal for which the image processes have been performed to the display apparatus 5041. Further, the CCU 5039 transmits a control signal to the camera head 5005 to control driving of the camera head 5005. The control signal may include information relating to an image pickup condition such as a magnification or a focal distance.

The display apparatus 5041 displays an image based on an image signal for which the image processes have been performed by the CCU 5039 under the control of the CCU 5039. If the endoscope 5001 is ready for imaging of a high resolution such as 4K (horizontal pixel number 3840×vertical pixel number 2160), 8K (horizontal pixel number 7680×vertical pixel number 4320) or the like and/or ready for 3D display, then a display apparatus by which corresponding display of the high resolution and/or 3D display are possible may be used as the display apparatus 5041. Where the apparatus is ready for imaging of a high resolution such as 4K or 8K, if the display apparatus used as the display apparatus 5041 has a size of equal to or not less than 55 inches, then a more immersive experience can be obtained. Further, a plurality of display apparatus 5041 having different resolutions and/or different sizes may be provided in accordance with purposes.

The light source apparatus 5043 includes a light source such as, for example, a light emitting diode (LED) and supplies irradiation light for imaging of a surgical region to the endoscope 5001.

The arm controlling apparatus 5045 includes a processor such as, for example, a CPU and operates in accordance with a predetermined program to control driving of the arm unit 5031 of the supporting arm apparatus 5027 in accordance with a predetermined controlling method.

An inputting apparatus 5047 is an input interface for the endoscopic surgery system 5000. A user can perform inputting of various kinds of information or instruction inputting to the endoscopic surgery system 5000 through the inputting apparatus 5047. For example, the user would input various kinds of information relating to surgery such as physical information of a patient, information regarding a surgical procedure of the surgery and so forth through the inputting apparatus 5047. Further, the user would input, for example, an instruction to drive the arm unit 5031, an instruction to change an image pickup condition (type of irradiation light, magnification, focal distance or the like) by the endoscope 5001, an instruction to drive the energy treatment tool 5021 or the like through the inputting apparatus 5047.

The type of the inputting apparatus 5047 is not limited and may be that of any one of various known inputting apparatus. As the inputting apparatus 5047, for example, a mouse, a keyboard, a touch panel, a switch, a foot switch 5057 and/or a lever or the like may be applied. Where a touch panel is used as the inputting apparatus 5047, it may be provided on the display face of the display apparatus 5041.

Otherwise, the inputting apparatus 5047 is a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5047 includes a camera which can detect a motion of a user, and various kinds of inputting are performed in response to a gesture or a line of sight of a user detected from a video imaged by the camera. Further, the inputting apparatus 5047 includes a microphone which can collect the voice of a user, and various kinds of inputting are performed by voice collected by the microphone. By configuring the inputting apparatus 5047 such that various kinds of information can be inputted in a contactless fashion in this manner, especially a user who belongs to a clean area (for example, the surgeon 5067) can operate an apparatus belonging to an unclean area in a contactless fashion. Further, since the user can operate an apparatus without releasing a possessed surgical tool from its hand, the convenience to the user is improved.

A treatment tool controlling apparatus 5049 controls driving of the energy treatment tool 5021 for cautery or incision of a tissue, sealing of a blood vessel or the like. A pneumoperitoneum apparatus 5051 feeds gas into a body lumen of the patient 5071 through the pneumoperitoneum tube 5019 to inflate the body lumen in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

In the following, especially a characteristic configuration of the endoscopic surgery system 5000 is described in more detail.

(Supporting Arm Apparatus)

The supporting arm apparatus 5027 includes the base unit 5029 serving as a base, and the arm unit 5031 extending from the base unit 5029. In the example depicted, the arm unit 5031 includes the plurality of joint portions 5033a, 5033b and 5033c and the plurality of links 5035a and 5035b connected to each other by the joint portion 5033b. In FIG. 1, for simplified illustration, the configuration of the arm unit 5031 is depicted in a simplified form. Actually, the shape, number and arrangement of the joint portions 5033a to 5033c and the links 5035a and 5035b and the direction and so forth of axes of rotation of the joint portions 5033a to 5033c can be set suitably such that the arm unit 5031 has a desired degree of freedom. For example, the arm unit 5031 may preferably be configured such that it has a degree of freedom equal to or not less than 6 degrees of freedom. This makes it possible to move the endoscope 5001 freely within the movable range of the arm unit 5031. Consequently, it becomes possible to insert the lens barrel 5003 of the endoscope 5001 from a desired direction into a body lumen of the patient 5071.

An actuator is provided in each of the joint portions 5033a to 5033c, and the joint portions 5033a to 5033c are configured such that they are rotatable around predetermined axes of rotation thereof by driving of the respective actuators. The driving of the actuators is controlled by the arm controlling apparatus 5045 to control the rotational angle of each of the joint portions 5033a to 5033c thereby to control driving of the arm unit 5031. Consequently, control of the position and the posture of the endoscope 5001 can be implemented. Thereupon, the arm controlling apparatus 5045 can control driving of the arm unit 5031 by various known controlling methods such as force control or position control.

For example, if the surgeon 5067 suitably performs operation inputting through the inputting apparatus 5047 (including the foot switch 5057), then driving of the arm unit 5031 may be controlled suitably by the arm controlling apparatus 5045 in response to the operation input to control the position and the posture of the endoscope 5001. After the endoscope 5001 at the distal end of the arm unit 5031 is moved from an arbitrary position to a different arbitrary position by the control just described, the endoscope 5001 can be supported fixedly at the position after the movement. It is to be noted that the arm unit 5031 may be operated in a master-slave fashion. In this case, the arm unit 5031 may be remotely controlled by the user through the inputting apparatus 5047 which is placed at a place remote from the surgery room.

Further, where force control is applied, the arm controlling apparatus 5045 may perform power-assisted control to drive the actuators of the joint portions 5033a to 5033c such that the arm unit 5031 may receive external force by the user and move smoothly following the external force. This makes it possible to move, when the user directly touches with and moves the arm unit 5031, the arm unit 5031 with comparatively weak force. Accordingly, it becomes possible for the user to move the endoscope 5001 more intuitively by a simpler and easier operation, and the convenience to the user can be improved.

Here, generally in endoscopic surgery, the endoscope 5001 is supported by a medical doctor called scopist. In contrast, where the supporting arm apparatus 5027 is used, the position of the endoscope 5001 can be fixed more certainly without hands, and therefore, an image of a surgical region can be obtained stably and surgery can be performed smoothly.

It is to be noted that the arm controlling apparatus 5045 may not necessarily be provided on the cart 5037. Further, the arm controlling apparatus 5045 may not necessarily be a single apparatus. For example, the arm controlling apparatus 5045 may be provided in each of the joint portions 5033a to 5033c of the arm unit 5031 of the supporting arm apparatus 5027 such that the plurality of arm controlling apparatus 5045 cooperate with each other to implement driving control of the arm unit 5031.

(Light Source Apparatus)

The light source apparatus 5043 supplies irradiation light upon imaging of a surgical region to the endoscope 5001. The light source apparatus 5043 includes a white light source which includes, for example, an LED, a laser light source or a combination of them. In this case, where a white light source includes a combination of red, green, and blue (RGB) laser light sources, since the output intensity and the output timing can be controlled with a high degree of accuracy for each colour (each wavelength), adjustment of the white balance of a picked up image can be performed by the light source apparatus 5043. Further, in this case, if laser beams from the respective RGB laser light sources are irradiated time-divisionally on an observation target and driving of the image pickup elements of the camera head 5005 is controlled in synchronism with the irradiation timings, then images individually corresponding to the R, G and B colours can be picked up time-divisionally. According to the method just described, a colour image can be obtained even if a colour filter is not provided for the image pickup element.

Further, driving of the light source apparatus 5043 may be controlled such that the intensity of light to be outputted is changed for each predetermined time. By controlling driving of the image pickup element of the camera head 5005 in synchronism with the timing of the change of the intensity of light to acquire images time-divisionally and synthesizing the images, an image of a high dynamic range free from underexposed blocked up shadows and overexposed highlights can be created.

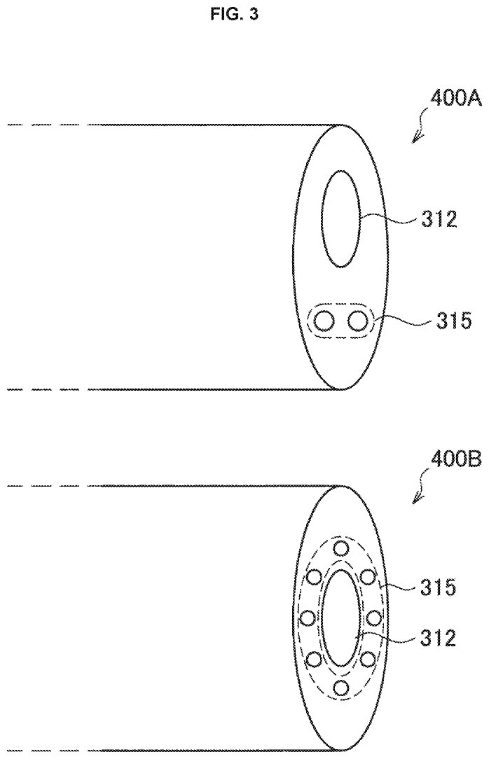

Further, the light source apparatus 5043 may be configured to supply light of a predetermined wavelength band ready for special light observation. This may include, but not be limited to laser light such as that provided by a Vertical Cavity surface laser or any kind of laser light. Alternatively or additionally, the light may be InfraRed (IR) light. In special light observation, for example, by utilizing the wavelength dependency of absorption of light in a body tissue to irradiate light of a narrower band in comparison with irradiation light upon ordinary observation (namely, white light), narrow band light observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 5043 can be configured to supply such narrow-band light and/or excitation light suitable for special light observation as described above. The light source may also apply a heat pattern to an area. This heat pattern will be explained later with reference to FIGS. 3A-C. The light source apparatus 5043 is, in embodiments, a Vertical Cavity Surface-Emitting Laser (VCSEL) which can produce light in the visible part of the electromagnetic spectrum and some produce light in the Infra-Red part of the electromagnetic spectrum. In this respect, the light source apparatus 5043 may also act as a visible light source illuminating the area. The light source apparatus 5043 is, in embodiments, one or more Vertical Cavity Surface-Emitting Laser (VCSEL) which can produce light in the visible part of the electromagnetic spectrum and some produce light in the Infra-Red part of the electromagnetic spectrum. In this respect, the light source apparatus 5043 may also act as a visible light source illuminating the area. The one or more VCSELs may be single wavelength narrowband VCSELs, where each VCSEL varies in emission spectral frequency. Alternatively, or additionally, one or more of the VCSELs may be a Micro Electro Mechanical system (MEMs) type VCSEL whose wavelength emission may be altered over a specific range. In embodiments of the disclosure, the wavelength may alter over the range 550 nm to 650 nm or 600 nm to 650 nm. The shape of the VCSEL may vary such as a square or circular shape and may be positioned at one or varying positions in the endoscope 5001.

The light source apparatus 5043 may illuminate one or more areas. This may be achieved by selectively switching the VCSELs on or by performing a raster scan of the area using a Micro Electro Mechanical system (MEMs). The purpose of the light source apparatus 5043 is to perform Spatial Light Modulation (SLM) on the light over the area. This will be explained in more detail later.

It should be noted that although the foregoing describes the light source apparatus 5043 as being positioned in the cart, the disclosure is not so limited. In particular, the light source apparatus may be positioned in the camera head 5005.

(Camera Head and CCU)

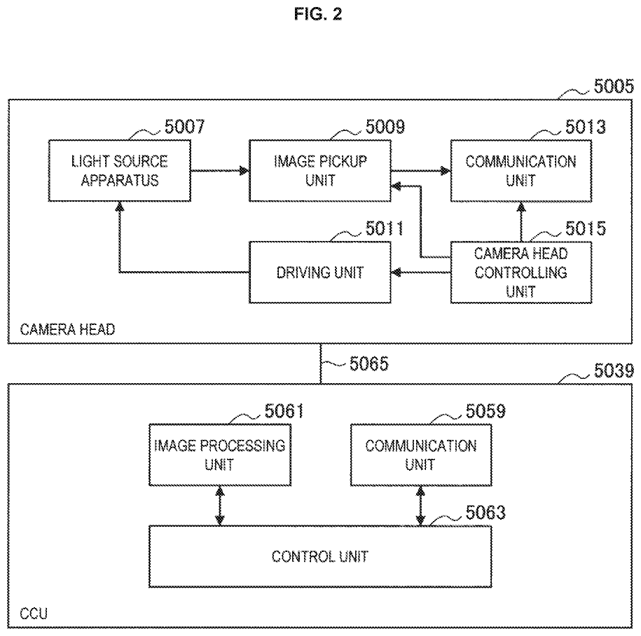

Functions of the camera head 5005 of the endoscope 5001 and the CCU 5039 are described in more detail with reference to FIG. 2. FIG. 2 is a block diagram depicting an example of a functional configuration of the camera head 5005 and the CCU 5039 depicted in FIG. 1.

Referring to FIG. 2, the camera head 5005 has, as functions thereof, a lens unit 5007, an image pickup unit 5009, a driving unit 5011, a communication unit 5013 and a camera head controlling unit 5015. Further, the CCU 5039 has, as functions thereof, a communication unit 5059, an image processing unit 5061 and a control unit 5063. The camera head 5005 and the CCU 5039 are connected to be bidirectionally communicable to each other by a transmission cable 5065.

First, a functional configuration of the camera head 5005 is described. The lens unit 5007 is an optical system provided at a connecting location of the camera head 5005 to the lens barrel 5003. Observation light taken in from a distal end of the lens barrel 5003 is introduced into the camera head 5005 and enters the lens unit 5007. The lens unit 5007 includes a combination of a plurality of lenses including a zoom lens and a focusing lens. The lens unit 5007 has optical properties adjusted such that the observation light is condensed on a light receiving face of the image pickup element of the image pickup unit 5009. Further, the zoom lens and the focusing lens are configured such that the positions thereof on their optical axis are movable for adjustment of the magnification and the focal point of a picked up image.

The image pickup unit 5009 includes an image pickup element and disposed at a succeeding stage to the lens unit 5007. Observation light having passed through the lens unit 5007 is condensed on the light receiving face of the image pickup element, and an image signal corresponding to the observation image is generated by photoelectric conversion of the image pickup element. The image signal generated by the image pickup unit 5009 is provided to the communication unit 5013.

As the image pickup element which is included by the image pickup unit 5009, an image sensor, for example, of the complementary metal oxide semiconductor (CMOS) type is used which has a Bayer array and is capable of picking up an image in colour. It is to be noted that, as the image pickup element, an image pickup element may be used which is ready, for example, for imaging of an image of a high resolution equal to or not less than 4K. If an image of a surgical region is obtained in a high resolution, then the surgeon 5067 can comprehend a state of the surgical region in enhanced details and can proceed with the surgery more smoothly.

Further, the image pickup element which is included by the image pickup unit 5009 includes such that it has a pair of image pickup elements for acquiring image signals for the right eye and the left eye compatible with 3D display. Where 3D display is applied, the surgeon 5067 can comprehend the depth of a living body tissue in the surgical region more accurately. It is to be noted that, if the image pickup unit 5009 is configured as that of the multi-plate type, then a plurality of systems of lens units 5007 are provided corresponding to the individual image pickup elements of the image pickup unit 5009.

The image pickup unit 5009 may not necessarily be provided on the camera head 5005. For example, the image pickup unit 5009 may be provided just behind the objective lens in the inside of the lens barrel 5003.

The driving unit 5011 includes an actuator and moves the zoom lens and the focusing lens of the lens unit 5007 by a predetermined distance along the optical axis under the control of the camera head controlling unit 5015. Consequently, the magnification and the focal point of a picked up image by the image pickup unit 5009 can be adjusted suitably.

The communication unit 5013 includes a communication apparatus for transmitting and receiving various kinds of information to and from the CCU 5039. The communication unit 5013 transmits an image signal acquired from the image pickup unit 5009 as RAW data to the CCU 5039 through the transmission cable 5065. Thereupon, in order to display a picked up image of a surgical region in low latency, preferably the image signal is transmitted by optical communication. This is because, upon surgery, the surgeon 5067 performs surgery while observing the state of an affected area through a picked up image, it is demanded for a moving image of the surgical region to be displayed on the real time basis as far as possible in order to achieve surgery with a higher degree of safety and certainty. Where optical communication is applied, a photoelectric conversion module for converting an electric signal into an optical signal is provided in the communication unit 5013. After the image signal is converted into an optical signal by the photoelectric conversion module, it is transmitted to the CCU 5039 through the transmission cable 5065.

Further, the communication unit 5013 receives a control signal for controlling driving of the camera head 5005 from the CCU 5039. The control signal includes information relating to image pickup conditions such as, for example, information that a frame rate of a picked up image is designated, information that an exposure value upon image picking up is designated and/or information that a magnification and a focal point of a picked up image are designated. The communication unit 5013 provides the received control signal to the camera head controlling unit 5015. It is to be noted that also the control signal from the CCU 5039 may be transmitted by optical communication. In this case, a photoelectric conversion module for converting an optical signal into an electric signal is provided in the communication unit 5013. After the control signal is converted into an electric signal by the photoelectric conversion module, it is provided to the camera head controlling unit 5015.

It is to be noted that the image pickup conditions such as the frame rate, exposure value, magnification or focal point are set automatically by the control unit 5063 of the CCU 5039 on the basis of an acquired image signal. In other words, an auto exposure (AE) function, an auto focus (AF) function and an auto white balance (AWB) function are incorporated in the endoscope 5001.

The camera head controlling unit 5015 controls driving of the camera head 5005 on the basis of a control signal from the CCU 5039 received through the communication unit 5013. For example, the camera head controlling unit 5015 controls driving of the image pickup element of the image pickup unit 5009 on the basis of information that a frame rate of a picked up image is designated and/or information that an exposure value upon image picking up is designated. Further, for example, the camera head controlling unit 5015 controls the driving unit 5011 to suitably move the zoom lens and the focus lens of the lens unit 5007 on the basis of information that a magnification and a focal point of a picked up image are designated. The camera head controlling unit 5015 may further include a function for storing information for identifying the lens barrel 5003 and/or the camera head 5005.

It is to be noted that, by disposing the components such as the lens unit 5007 and the image pickup unit 5009 in a sealed structure having high airtightness and waterproof, the camera head 5005 can be provided with resistance to an autoclave sterilization process.

Now, a functional configuration of the CCU 5039 is described. The communication unit 5059 includes a communication apparatus for transmitting and receiving various kinds of information to and from the camera head 5005. The communication unit 5059 receives an image signal transmitted thereto from the camera head 5005 through the transmission cable 5065. Thereupon, the image signal may be transmitted preferably by optical communication as described above. In this case, for the compatibility with optical communication, the communication unit 5059 includes a photoelectric conversion module for converting an optical signal into an electric signal. The communication unit 5059 provides the image signal after conversion into an electric signal to the image processing unit 5061.

Further, the communication unit 5059 transmits, to the camera head 5005, a control signal for controlling driving of the camera head 5005. The control signal may also be transmitted by optical communication.

The image processing unit 5061 performs various image processes for an image signal in the form of RAW data transmitted thereto from the camera head 5005. The image processes include various known signal processes such as, for example, a development process, an image quality improving process (a bandwidth enhancement process, a super-resolution process, a noise reduction (NR) process and/or an image stabilization process) and/or an enlargement process (electronic zooming process). Further, the image processing unit 5061 performs a detection process for an image signal in order to perform AE, AF and AWB.

The image processing unit 5061 includes a processor such as a CPU or a GPU, and when the processor operates in accordance with a predetermined program, the image processes and the detection process described above can be performed. It is to be noted that, where the image processing unit 5061 includes a plurality of GPUs, the image processing unit 5061 suitably divides information relating to an image signal such that image processes are performed in parallel by the plurality of GPUs.

The control unit 5063 performs various kinds of control relating to image picking up of a surgical region by the endoscope 5001 and display of the picked up image. For example, the control unit 5063 generates a control signal for controlling driving of the camera head 5005. Thereupon, if image pickup conditions are inputted by the user, then the control unit 5063 generates a control signal on the basis of the input by the user. Alternatively, where the endoscope 5001 has an AE function, an AF function and an AWB function incorporated therein, the control unit 5063 suitably calculates an optimum exposure value, focal distance and white balance in response to a result of a detection process by the image processing unit 5061 and generates a control signal.

Further, the control unit 5063 controls the display apparatus 5041 to display an image of a surgical region on the basis of an image signal for which image processes have been performed by the image processing unit 5061. Thereupon, the control unit 5063 recognizes various objects in the surgical region image using various image recognition technologies. For example, the control unit 5063 can recognize a surgical tool such as forceps, a particular living body region, bleeding, mist when the energy treatment tool 5021 is used and so forth by detecting the shape, colour and so forth of edges of the objects included in the surgical region image. The control unit 5063 causes, when it controls the display unit 5041 to display a surgical region image, various kinds of surgery supporting information to be displayed in an overlapping manner with an image of the surgical region using a result of the recognition. Where surgery supporting information is displayed in an overlapping manner and presented to the surgeon 5067, the surgeon 5067 can proceed with the surgery more safety and certainty.

The transmission cable 5065 which connects the camera head 5005 and the CCU 5039 to each other is an electric signal cable ready for communication of an electric signal, an optical fibre ready for optical communication or a composite cable ready for both of electrical and optical communication.

Here, while, in the example depicted, communication is performed by wired communication using the transmission cable 5065, the communication between the camera head 5005 and the CCU 5039 may be performed otherwise by wireless communication. Where the communication between the camera head 5005 and the CCU 5039 is performed by wireless communication, there is no necessity to lay the transmission cable 5065 in the surgery room. Therefore, such a situation that movement of medical staff in the surgery room is disturbed by the transmission cable 5065 can be eliminated.

An example of the endoscopic surgery system 5000 to which the technology according to an embodiment of the present disclosure can be applied has been described above. It is to be noted here that, although the endoscopic surgery system 5000 has been described as an example, the system to which the technology according to an embodiment of the present disclosure can be applied is not limited to the example. For example, the technology according to an embodiment of the present disclosure may be applied to a soft endoscopic system for inspection or a microscopic surgery system. Indeed, the technology may be applied to a surgical microscope for conducting neurosurgery or the like. Moreover, the technology may be applied more generally to any kind of medical imaging.

The technology according to an embodiment of the present disclosure can be applied suitably to the CCU 5039 from among the components described hereinabove. Specifically, the technology according to an embodiment of the present disclosure is applied to an endoscopy system, surgical microscopy or medical imaging. By applying the technology according to an embodiment of the present disclosure to these areas, blood flow in veins, arteries and capillaries may be identified. Further, objects may be identified and the material of those objects may be established. This reduces the risk to the patient's safety during operations.

The light source apparatus 5043 is, in embodiments, one or more Vertical Cavity Surface-Emitting Laser (VCSEL) which can produce light in the visible part of the electromagnetic spectrum and some produce light in the Infra-Red part of the electromagnetic spectrum. In this respect, the light source apparatus 5043 may also act as a visible light source illuminating the area. The one or more VCSELs may be single wavelength narrowband VCSELs, where each VCSEL varies in emission spectral frequency. Alternatively, or additionally, one or more of the VCSELs may be a Micro Electro Mechanical system (MEMs) type VCSEL whose wavelength emission may be altered over a specific range. In embodiments of the disclosure, the wavelength may alter over the range 550 nm to 650 nm or 600 nm to 650 nm. The shape of the VCSEL may vary such as a square or circular shape and may be positioned at one or varying positions in the endoscope system 5000.

The light source apparatus 5043 may illuminate one or more areas and/or objects within the areas. This may be achieved by selectively switching the VCSELs on or by performing a raster scan of the area using a Micro Electro Mechanical system (MEMs). The purpose of the light source apparatus 5043 is to perform Spatial Light Modulation (SLM) on the light over the area. This will be explained in more detail later.

Referring to FIG. 3, two particular embodiments describing the relationship between the lens arrangement in the image pickup unit 5009 and the light source apparatus 5043 is shown. However, as would be understood, the arrangement is not limiting and is exemplary only. In a first embodiment, an end 400A of the camera head 5005 is shown. The end 400A has the lens arrangement positioned above the light source apparatus 5043. Of course, the disclosure is not so limited and the light source apparatus 5043 may be positioned below the lens arrangement or to the left or right of the lens arrangement or in some way offset from the lens arrangement. In other words, the light source apparatus 5043 is positioned adjacent to the lens arrangement. The light source apparatus 5043 may have one VCSEL positioned at one position and a second VCSEL positioned at a second position relative to the lens arrangement. For example, the first and second VCSEL may be positioned at opposite sides of the lens arrangement. In other words, the first and second VCSEL may be separated by 180°. Of course, the disclosure is not so limited and the first and second VCSEL may be positioned relative to one another with respect to the lens arrangement.

The light source apparatus 5043 in this arrangement includes two horizontally displaced VCSELs and is shown with a dashed line. Of course, it is envisaged that more than or less than two VCSELs may be provided. These VCSELs may be narrow band VCSELs or may have a varying emission range.

In the embodiment, the two VCSELs are each controlled independently of one another. The two VCSELs are directed to an area such that if one VCSEL is illuminated the area is illuminated from one direction and if the second VCSEL is illuminated, the same area is illuminated from a different direction. This allows for light source direction variation enabling different areas to be illuminated and spatial variation as will now be described.

a. The spatial variance allows different angles of incidence of the illuminating light on the area to be used (by the sources of illumination being placed in different positions on the endoscope). This creates both shadows within the scene and different intensity gradients on the viewed scene. These both depend on the positions and angles of the surfaces and objects with respect to the light sources. This can be analysed to provide topographic and curvature information as will be explained later.

b. The spatial variation, which can be implemented by redirecting the VCSEL laser light generated by one of the VCSEL devices off a MEMs actuated micromirror or any kind of structure having a reflective surface which allows the light to be redirected, or by actuating a platform on which the VCSEL is mounted, allows specific parts of the image to be illuminated in light (for example of a selected wavelength) or for the light beam to be scanned across the area of interest in a raster or spiral scanning pattern, thereby revealing detailed information on a part of the image. This will be described later.

As noted above, the light source apparatus 5043 may have various arrangements. Embodiments are shown with a second end arrangement 400B where the light source apparatus 5043 consists of a plurality of VCSELs surrounding the lens arrangement.

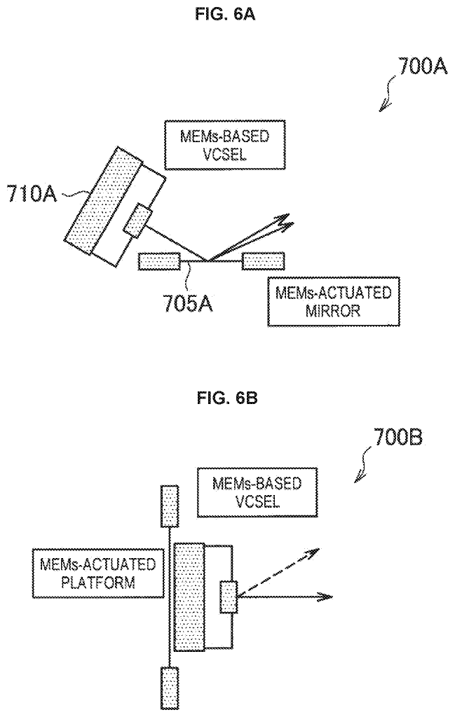

The advantages of the spatial variance and the spatial variation are explained with reference to FIGS. 6 to 14.

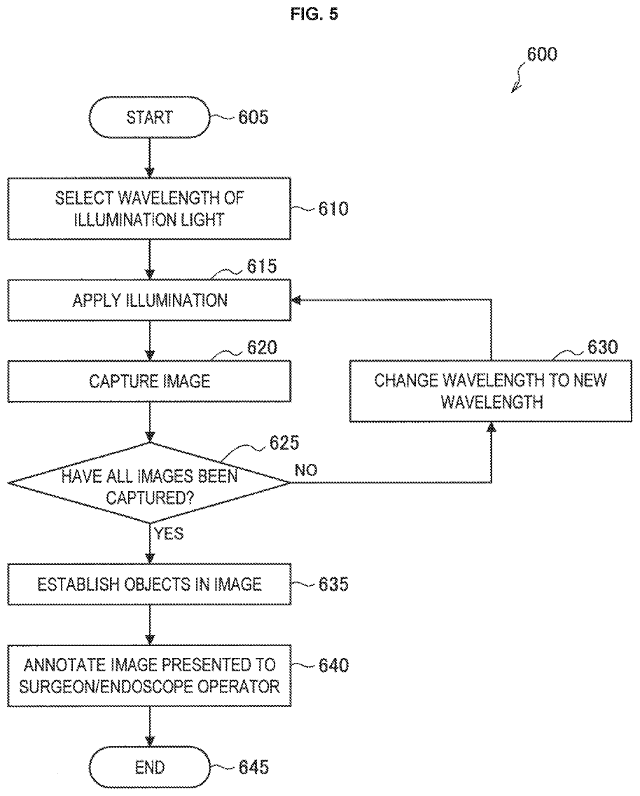

Referring to FIG. 5, a graph 500 shows the Molar extinction coefficient versus wavelength for deoxygenated haemoglobin in line 505 and oxygenated haemoglobin in line 510. In other words, the graph 500 shows the light absorption for deoxygenated and oxygenated haemoglobin.

From graph 500, the spectrum of haemoglobin shows a sharp change at around 600 nm for both oxygenated and deoxygenated variants. Therefore, it is possible to identify the presence of haemoglobin within the area by looking at the difference in light absorption between narrow band light at around 600 nm and at around 650 nm. These differences reveal the presence of blood vessels containing haemoglobin since light at these wavelengths penetrates tissues reasonably well.

Therefore, the light source apparatus 5043 is controlled by the CCU 5039 to perform a spectral variance. This allows the scene (or part of a scene) to be illuminated by light with a single, narrow frequency band. By selecting a set of appropriate narrow band light sources, or by modulating a single MEMs-based VCSEL to emit light in a range of frequencies, and sequencing through this set of single narrowband illumination sources, accurate data can be gathered concerning the colour of objects within the scene (or part of a scene) even when those differences are small. This can be especially important in detecting blood vessels below the surface as haemoglobin has significant differences in absorption at close frequencies, such as at 600 and 630 nm where the former is absorbed and the latter significantly less.

By detecting the presence of blood vessels within an image, it is possible for the surgeon and/or the endoscope operator to avoid rupturing or otherwise damaging tissue. In order to achieve this, the CCU 5039 controls the light source apparatus 5043 and the image pickup unit 5009 to perform a process set out in FIG. 5. Specifically, the light source apparatus 5043 illuminates an area with light at a particular wavelength and the image pickup unit 5009 captures this image. The wavelength of the light source apparatus 5043 is then changed (either by activating a different VCSEL or by varying the emission wavelength of a single VCSEL) and the area illuminated. The image pickup unit 5009 then captures this image.

By then comparing the relative brightness of the pixels in the two images, an overlay image can be produced which uses the brightness differences between the tissue reflections of the different wavelength illuminators to show small colour differences in the underlying tissues. The overlay image is provided on the conventionally captured image to highlight the location of the blood vessels. These brightness differences identify the difference in absorption of the haemoglobin.

FIG. 5 shows a flow chart 600 explaining this process in detail.

The process starts at step 605. In step 610, the first wavelength of the light source apparatus 5043 is selected. In embodiments, the range of wavelengths will be selected where there is a sharp change in absorption of light within haemoglobin. In embodiments, the range is between 600 nm and 650 nm. Of course other ranges are envisaged, such as between 400 nm and 500 nm where there is a sharp change in absorption, but 600 nm to 650 nm is preferable due to the sharpness of the change. In this preferable range, therefore, a wavelength of 600 nm is selected.

The process moves to step 615 where the light source apparatus 5043 illuminates the area with the selected wavelength of light. The image pickup unit 5009 captures the image of the area illuminated with the light in step 620.

The process then moves to step 625 where a decision is made whether all the images have been captured. This decision is made based upon whether the light source apparatus 5043 has illuminated the area at all the wavelengths in the range. In other words, a decision is made whether the light source apparatus 5043 has illuminated the area for all wavelengths. Of course, other determining factors are envisaged such as whether a predetermined number of images have been captured.

If the decision is made that not all images have been captured, the no path is followed and the process moves to step 630.

In step 630, the wavelength of the light source apparatus 5043 is changed. In embodiments, this may mean a second narrow bandwidth VCSEL is activated to illuminate the area. Alternatively, the emission wavelength of a varying VCSEL is changed. The wavelength may be changed by 10 nm, 20 nm or any non-overlapping value. Indeed, the value may not change linearly. For example, the amount of change of the wavelength values may vary non-linearly such that the change in absorption is linear. In other words, for wavelengths where there is significant change in absorption, a small change in wavelength may be made.

The process then returns to step 615.

On the other hand, if at step 625 the decision is made that all the images have been captured, the yes path is followed.

The process moves to step 635 where the objects in the image are established. Specifically, in this case, the blood vessels within the image are established. In order to achieve this, the images captured at each wavelength are corrected for the raw intensity of the light source and corrected for any differences resulting from different positions of the light source, or movement of the camera head 5005. This processing is performed to normalise the images so that the only difference between images results from the absorption of the light. This is performed using known techniques.

The relative brightness of the pixels in the set of images is then compared. In other words, the brightness of each pixel in one image is compared with the brightness of each pixel in each of the other images. This provides a map where for each pixel in the image, the brightness across the range of wavelengths is derived. Accordingly, the light absorption at each pixel position is determined.

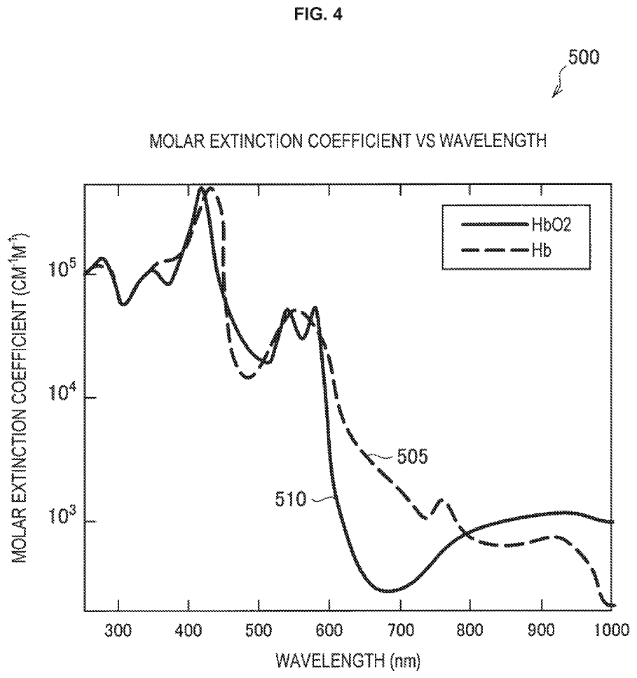

By determining the light absorption at each pixel position for each wavelength across the range of wavelengths, the CCU 5039 determines the material at that pixel position. In particular, although not limited, the CCU 5039 determines the presence of haemoglobin using the absorption table in FIG. 4. This identifies the presence of blood and blood vessels.

The CCU 5039 then provides an overlay image which is a graphic where the material at each pixel position is identified. This clearly highlights the location of the haemoglobin at each respective pixel position.

The process moves to step 640 where an image from the endoscope is overlaid with the overlay image. In other words, the conventionally captured image which is displayed to the surgeon or endoscopy operator and is formed of pixels corresponding to the pixels captured using the technique of FIG. 5 is annotated with the location of the haemoglobin. Of course, although the above describes creating an overlay image, the disclosure is not so limited and the captured image may be annotated using the technique of FIG. 5.

This embodiment enables the endoscope operator or surgeon to more clearly define the location of haemoglobin (or other relevant fluid or material) and thus reduce the risk of injury to the patient. This is because the reflections from the tissue when different wavelength light is used to illuminate the tissue emphasises small colour differences in the image.

Although the above describes the brightness levels of each pixel being compared for each image having illumination at a particular wavelength, the disclosure is not so limited. For example, a weighting could be applied to pixels from images captured using different wavelengths. The choice of weighting may be dependent on the material to be emphasised. For example, if the embodiment of the disclosure is configured to detect haemoglobin, a high weighting may be applied at 650 nm where the absorption of light at that wavelength is low. Similarly, for images captured with an illumination of 600 nm, where the absorption of haemoglobin is quite high (compared with the absorption at 650 nm), a low weighting may be applied. This emphasises the presence of haemoglobin.

In addition, to further accentuate the visibility of blood vessels, the area may initially be illuminated with a light at 540 nm and an image captured. This will provide a benchmark image whose wavelength of illumination is selected to correct for general reflectance (540 nm is particularly well absorbed by blood). The captured images having the varying wavelength illumination may be first divided by the reference image to accentuate the visibility of blood vessels before being compared with one another.

In addition, the brightness of the irradiating light may be controlled to reduce the amount of undesirable reflection from the surface of the tissue. In particular, in order to reduce glare from the irradiating light, the brightness of the irradiating light may be controlled in accordance with the distance between the VCSEL and the tissue. Specifically, the brightness of the VCSEL is reduced when the distance between the VCSEL and the tissue is less than a predetermined distance. This reduces the amount of glare from the reflection of the VCSEL on the tissue when the VCSEL is close to the tissue. The material may then be determined as described above when the brightness of the VCSEL is adjusted to remove any undesirable reflections.

Referring to FIGS. 6A and 6B, a system according to embodiments of the disclosure is described. As mentioned above, in the section noting spatial variance and spatial variation, it is useful to illuminate an area of interest during endoscopy from several directions. The illumination may be from different directions of incidence (as explained with regard to FIG. 3) or may direct light to specific areas (or even provide a scan pattern such as a raster or spiral scan) thus allowing other areas to be illuminated without moving the endoscope head. This allows the curvature of the surface to be calculated by using Photometric Stereo and/or determine the shadow topography which allows the shape and position of the object with respect to the background.

The system described in FIGS. 6A and 6B may also be used to illuminate the area with the varying wavelength light described with reference to FIG. 5.

For example, in a non-limiting embodiment, the brightness of the VCSEL may be adjusted to reduce the undesirable reflection of light from the surface of the area. Specifically, once the structure or topography of the object is detected, appropriate lighting control is achieved based on the distance of the VCSEL to the object. In addition or alternatively, the lighting condition may be achieved based on the structure or topography itself.

As an example of the above, at the area close to the endoscope, weak light is irradiated to avoid undesirable reflection of the light and at an area far from the endoscope, the intensity of the light is increased as the probability of undesirable reflections is reduced.

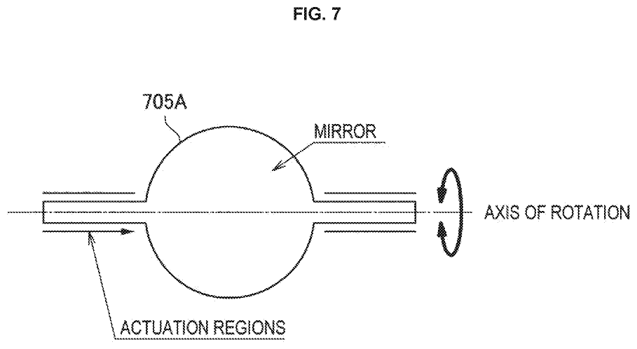

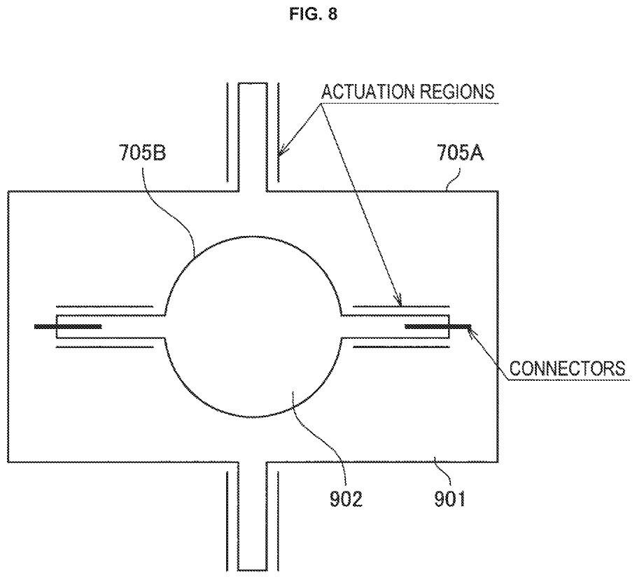

In FIG. 6A, a first system 700A is shown. In this system 700A, a single VCSEL is provided. In particular, the MEMs based VCSEL 710A used to illuminate the area is provided. The MEMs based VCSEL 710A is directed toward a MEMs actuated mirror 705A. Embodiments of the MEMs actuated minor 705A are shown in FIGS. 7 and 8. It should be noted that a plurality of the systems described in FIGS. 6A and 6B may be provided to illuminate an area from a number of different directions.

In FIG. 7, the MEMs actuated mirror 705A rotates around a single axis of rotation (depicted by the dotted line). This allows the minor to reflect light along a single scan line. In order to perform raster scanning of an area, the MEMs device upon which the mirror is mounted will also move to allow the scan line to move from one side of the area to the other.

In FIG. 8, the MEMs actuated mirror 705B rotates around two axes of rotation. In particular, a first gimbal 901 rotates about a first axis and a second gimbal 902 rotates about a second axis; the second axis being orthogonal to the first axis. The second gimbal 902 has a mirrored surface or a mirror located thereon. The first gimbal 901 may or may not have a mirrored surface. This allows the MEMs actuated minor 705A to perform raster scanning of an area by moving the second gimbal 902 along the single scan line as noted in FIG. 7. The first gimbal 902 which is fixedly connected to the second gimbal 901 moves orthogonally in respect of the first gimbal 902 so that raster scanning is performed.

The MEMs actuated minor 705A and 705B described in FIGS. 7 and 8 respectively are controlled by the CCU 5039.

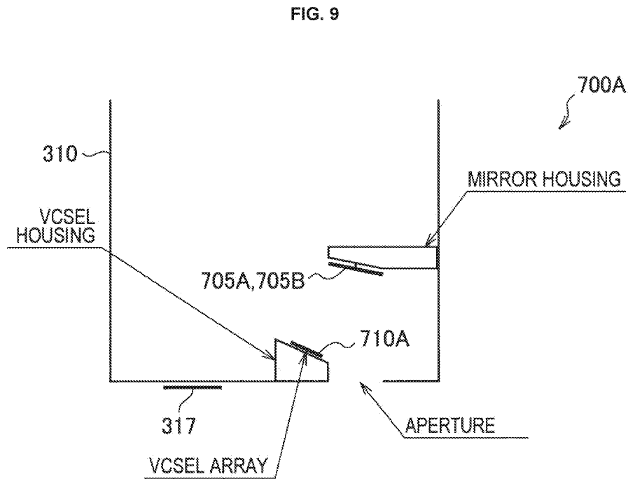

In FIG. 9 a camera head 5005 according to embodiments is shown. In this example, the MEMs actuated minor 705A or 705B of FIGS. 7 and 8 is mounted on a mirror housing. The minor housing may move or not depending upon whether a raster scan is required and whether the mirror mounted thereon in the minor 705A of FIG. 7 or mirror 705B of FIG. 8. In other words, in the event that the mirror 705A of FIG. 7 is mounted, the mirror housing will rotate in an orthogonal axis compared to the axis of mirror 705A and in the event that the minor 705B of FIG. 8 is mounted, the mirror housing will not need to rotate.

Additionally positioned in the camera head 5005 is the VCSEL 710A. In embodiments this may be an array of VCSELs emitting light of the same or different wavelengths. Alternatively, the VCSEL may emit light of varying wavelengths. Of course, rather than an array of VCSELs, a single VCSEL may be provided. The housing for the VCSEL is fixed.

The image sensor is part of the image pickup unit 5009 which is part of the camera head 5005. It is envisaged that the image sensor may be instead placed anywhere appropriate within the endoscope system 5000.

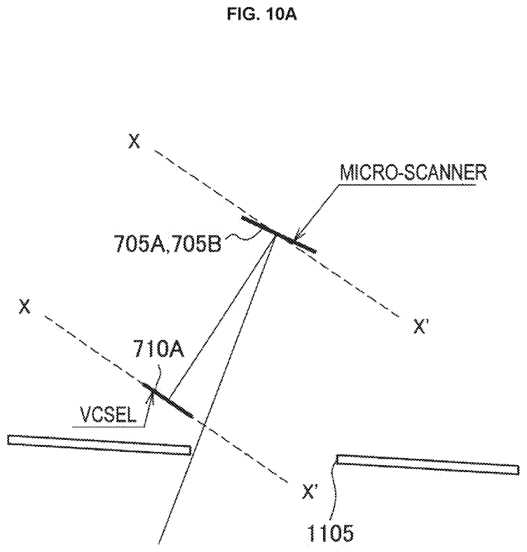

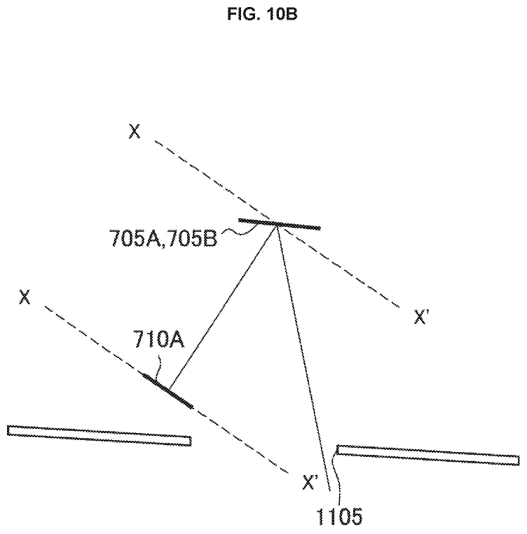

The operation of the system will generally be described with reference to FIG. 10A and 10B. In particular, the VCSEL 710A is activated sending light to the MEMs actuated mirror 705A and 705B. The light generated by the VCSEL is reflected off the mirrored surface of the mirror 705A and 705B. The CCU 5039 then controls the movement of the mirror 705A and 705B to direct the light through the endoscope aperture 1105. The CCU 5039 then controls the mirror 705A or 705B to scan the reflected light along a scan line.

The CCU 5039 then controls the minor 705B or the mirror mount (in the case of the mirror being 705A) to perform a raster scan. This illuminates a large area (which may include an object of interest) within the patient. Of course, any kind of scan or movement is envisaged and may be selected depending upon the endoscope operator or surgeon's preference. For example, the mirror or mirror mount may be controlled to simply direct the light emanating from the VCSEL to a particular area within the patient.

The image sensor within the image pickup unit 5009 may then capture one or more images as required. As noted above, systems of FIG. 10A and 10B may operate according to the principles of the earlier embodiment described with reference to FIG. 5 to more accurately identify blood vessels. Alternatively, the systems of FIGS. 10A and 10B may operate independently of the earlier embodiments.

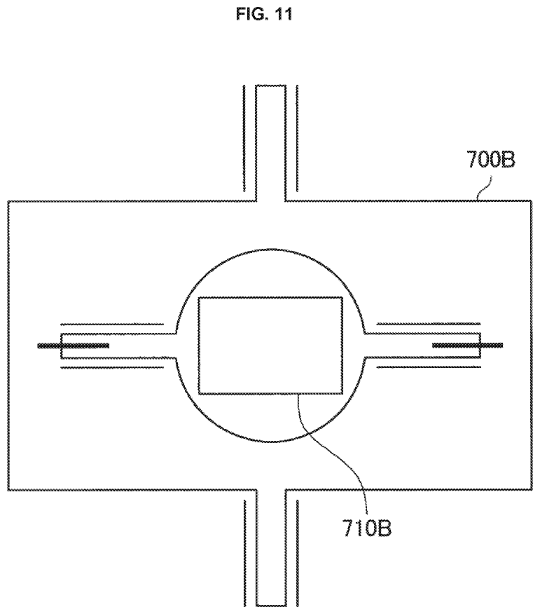

In FIG. 11, the system of FIG. 6B is shown in more detail. Specifically, similarly to the system of FIG. 8 where the mirror 902 is placed on the second gimbal, instead the VCSEL or array of VCSELs 710B is placed on the MEMs actuated platform. This allows the VCSEL to rotate about two orthogonal axes and direct the emitted light in an appropriate manner. In this system, as no reflective surface or minor is required, the size of the system shown in FIG. 11 is smaller than that described in FIG. 10A or 10B. This is advantageous for endoscopy where a small size endoscope head is desirable.

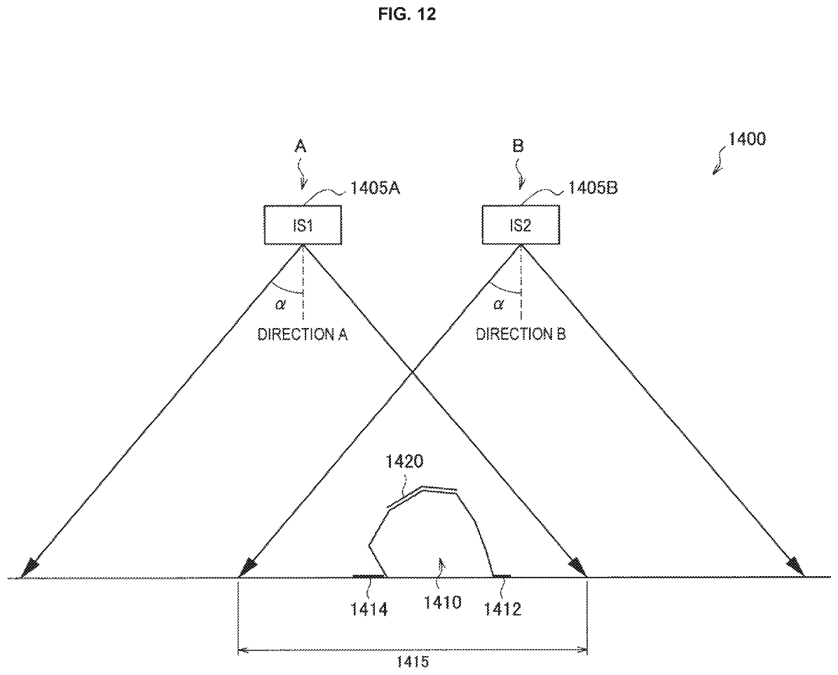

In FIG. 12, a system 1400 whereby two separate VCSELs 1405A and 1405B (or VCSEL and MEMs mirror/reflective structure combinations) provide light from different directions to illuminate an area is described. It is noted here that the VCSEL light may be directly provided by individual VCSELs as in the case of FIG. 3 or may be provided using the one of the arrangements described in FIGS. 6A or 6B. In the event that the VCSEL light is scanned across the object 1410 by the arrangement of FIGS. 6A or 6B, the value of a is changed as the illumination is scanned.

In this system 1400, a first VCSEL 1405A illuminates object 1410 from a first direction (direction A) and a second VCSEL 1405B illuminates object 1410 from a second direction (direction B). As is evident from FIG. 12, the illumination from the first VCSEL 1405A and the second VCSEL 1405B overlap in area 1415. The illumination, when provided from direction A, casts a first shadow 1412 and the illumination, when provided from direction B, casts a second shadow 1414. Obviously, although the foregoing describes two or more VCSELs or MEMs mirrors in FIG. 12, the disclosure is not so limited. In fact, more than two VCSELs or MEMs mirrors may be provided. Indeed, only one VCSEL or MEMs mirror may be provided if the VCSEL or MEMs mirror moves between two locations or if the MEMs mirror is so large that it may be illuminated at two different parts. The important point is that the object 1410 is illuminated from two or more different directions.

When illuminated from two or more different directions, photometric stereo is performed to resolve the surface angle 1420 in the object. This provides the topology information of the object. This is described in NPL 1. After the topography information has been established, this is overlaid onto the captured image from the endoscope and provided to the surgeon. This provides the surgeon with a better understanding of the topography of the tissue being viewed.

In addition, the wavelength of the light emanating from the VCSEL may be varied. As the transmittance of a material varies for different wavelength light, the material of the object may also be derived.

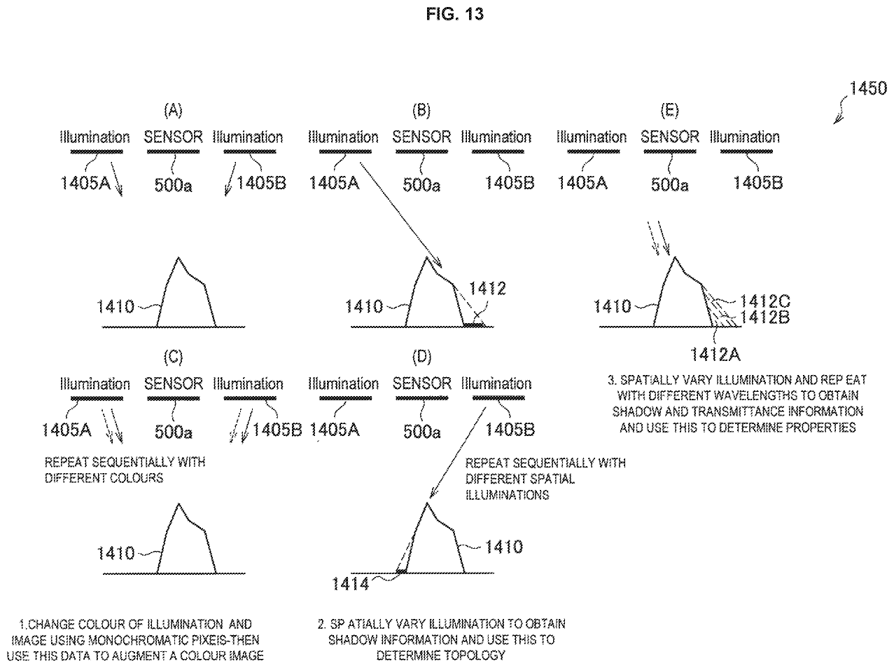

The process will be explained with reference to FIG. 13.

In FIG. 13, the sequence of illuminating the object 1410 of FIG. 12 is shown. In diagram (A), the object 1410 is illuminated by the first VCSEL 1405A and the second VCSEL 1405B. In diagram (B), the illumination from the first VCSEL 1405A casts a shadow 1412 which is captured by image sensor 5009. In diagram (C), the colour of the illumination provided by the first VCSEL 1405A is changed. By changing the colour of illumination and then capture the resulting image using monochromatic pixels, the photometric stereo process is more accurate as the impact of movement of the object is mitigated.

In diagram (D), the object 1410 is illuminated by the second VCSEL 1405B. This produces the second shadow 1414. Again the colour of the illumination provided by the first VCSEL 1405A is changed. After the sequence in diagram (D) has been completed, the topography of the object will be determined.

The process then moves to diagram (E), where the wavelengths of the illumination provided by the first VCSEL 1405A and the second VCSEL 1405B is changed. This change in wavelengths provides transmittance information associated with the object 1410. This transmittance information is compared with transmittance information associated with known materials to determine the tissue properties of the object 1410.

The topography information and the tissue properties are then overlaid on the image captured by the endoscope. In other words, the determined topography information and tissue information is provided to the surgeon by annotating the endoscope image displayed to the surgeon.

As noted above, the purpose of illuminating an area with light from different directions in endoscopy is to provide spatial variance so that light of different angles of incidence are used and direction variance where the light is directed to specific areas or to perform a scan, either raster scan or spiral scan or the like.

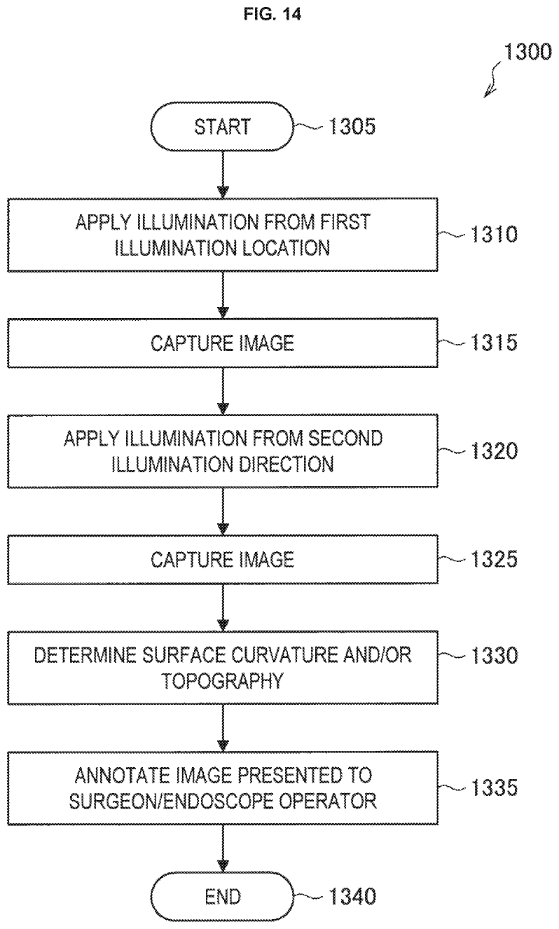

This process will now be described with reference to FIG. 14.

In FIG. 14, a flow chart 1300 showing a process according to embodiments of the disclosure is described. The process starts in step 1305.

The process then moves to step 1310 where the light emitted by the VCSEL is directed to an area in the patient. This illuminates the area from a first direction. The process then moves to step 1315 where the image sensor 317 captures the image.

The process moves to step 1320 where the light emitted by a VCSEL in a different position is directed to the area. This illuminates the area from the second direction. This is achieved by either moving the MEMs actuated mirror 705A or 705B (FIGS. 7 and 8) or by moving the mounting of the VCSEL (FIG. 11) or by activating a different VCSEL in an array of VCSELs.

The process moves to step 1325 where the image is captured.

The process moves to step 1330. The CCU 5039 determines either the surface curvature function or the shadow topography function.

In order to determine the surface curvature function, the CCU 5039 they are corrected for the raw intensity of the associated light source. The CCU 5039 then compares the brightness of each pixel in the captured images obtained from the different illumination source locations. By having the light source illuminate the area from a different direction the effect of the surface albedo and surface normal can be separated. In general, the intensity of reflected light from a diffuse surface depends on the albedo of the material and a function of the angle between the surface and angle to the light source. Using several light sources illuminating from different angles the two effects (albedo and surface normal) can be separated, by first deriving the surface normal, for example from a photometric stereo look-up table using the relative intensities of the same image pixels illuminated from different directions and a value for the material albedo which can be derived by correcting the image pixel value for the effects of the surface normal on reflectance.

In order to determine the shadow topography function, the first and second images are corrected for the raw intensity of the associated VCSEL. The shadows are recognized in the image and the shape and relationship between the object and background can be determined. If an object is nearer the light source than a background and surfaces between lie out of the path of the light a shadow will be cast by the object on the background. A part of an object nearer the light source with a concavity may cast a shadow on a part of the object that is further away. Depending on the direction of the illumination, shadows will appear at different positions. If the shadows are found, and the corresponding points across the shadow boundary correctly inferred, then information can be derived on the shape and position of the object with respect to the background. One method of using shadows to infer shapes and relative depths is ‘shadow carving’, an algorithm which iteratively recovers an estimate of the object from an object silhouette and its shadows which approximates the object's shape more and more closely as more illumination directions are added and is provably an outer bound to the object's shape. By using several different directions of illumination additional shadow constraints on shape can be obtained. This is described in NPL 2.

The CCU 5039 then provides an overlay image which is a graphic where the surface function and the topography is identified.

The process moves to step 1335 where an image from the endoscope is overlaid with the overlay image. In other words, the conventionally captured image which is displayed to the surgeon or endoscopy operator and is formed of pixels corresponding to the pixels captured using the technique of FIG. 14 is annotated with the surface function and/or the topography. Of course, although the above describes creating an overlay image, the disclosure is not so limited and the captured image may be annotated using the technique of FIG. 14.

The process ends in step 1340.

It will be understood that where a VCSEL is described, any light source is envisaged.

It will be understood that a wavelength and the angle of light emitted from the Vertical Cavity Surface Emitting Laser are examples of light conditions. In other words, a light condition is a characteristic of the light such as a physical characteristic (like a wavelength, brightness or intensity) or an angle of emittance of the light.

It will be understood that the material and the topography of an object are examples of information about the object. The information of the object may be therefore physical characteristics of the object.

Various embodiments of the present disclosure are defined by the following numbered clauses:

1. A medical imaging device comprising a Vertical Cavity Surface Emitting Laser configured to illuminate an object using one of a plurality of light conditions, an image sensor configured to capture an image of the object, and circuitry configured to:
control the Vertical Cavity Surface Emitting Laser to illuminate the object using light having a first light condition;
control the image sensor to capture a first image of the object with the light having the first light condition;
control the Vertical Cavity Surface Emitting Laser to illuminate the object with light having a second, different, light condition;
control the image sensor to capture a second image of the object illuminated with the light having the first light condition; and
determine information of the object based on the first image and the second image.
2. A device according to paragraph 1, wherein the light condition is a wavelength of the light.
3. A device according to paragraph 2, wherein the range of the wavelength is between 600 and 650 nm.
4. A device according to paragraph 1 or 2, wherein the first light condition is a wavelength of 540 nm.
5. A device according to paragraph 2 to 4, wherein the information of the object is the material of the object and that the material of the object is determined by comparing the brightness of the first and second image at corresponding points within the images.
6. A device according to any preceding paragraph, wherein the light condition is a brightness of the light.
7. A device according to any preceding paragraph, wherein the light condition is the angle of light emitted from the Vertical Cavity Surface Emitting Laser.
8. A device according to any preceding paragraph, wherein the first light condition is illuminating the object from a first direction and the second light condition is illuminating the object from a second, different, direction and the information of the object is the topography of the object, the topography being determined by photometric stereo.
9. A device according to any preceding paragraph, comprising a plurality of Vertical Cavity Surface Emitting Lasers.
10. A device according to any preceding paragraph, comprising a tip for entry into a patient, wherein the tip comprises the Vertical Cavity Surface Emitting Laser.
11. A device according to any preceding paragraph, comprising a lens arrangement to focus light onto the image sensor, wherein the Vertical Cavity Surface Emitting Laser is located adjacent to the lens arrangement.
12. A device according to paragraph 11 comprising a second Vertical Cavity Surface Emitting Laser located adjacent to the lens arrangement, on the side opposite the Vertical Cavity Surface Emitting Laser.
13. A device according to any preceding paragraph, comprising a structure having a reflective surface configured to change the direction of the light emitted by the Vertical Cavity Surface Emitting Laser.
14. A device according to paragraph 13, wherein the structure is a minor configured to move so that the reflected light is scanned over the object.
15. A device according to any preceding paragraph, wherein the control circuitry is further configured to: annotate an image of the object using the determined object information.
16. An endoscope comprising a device according to any preceding paragraph.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A medical imaging device comprising a Vertical Cavity Surface Emitting Laser configured to illuminate an object using one of a plurality of light conditions, an image sensor configured to capture an image of the object, and circuitry configured to:
control the Vertical Cavity Surface Emitting Laser to illuminate the object using light having a first light condition;
control the image sensor to capture a first image of the object with the light having the first light condition;
control the Vertical Cavity Surface Emitting Laser to illuminate the object with light having a second, different, light condition;
control the image sensor to capture a second image of the object illuminated with the light having the first light condition; and
determine information of the object based on the first image and the second image.

2. A device according to claim 1, wherein the light condition is a wavelength of the light.

3. A device according to claim 2, wherein the range of the wavelength is between 600 and 650 nm.

4. A device according to claim 2, wherein the first light condition is a wavelength of 540 nm.

5. A device according to claim 2, wherein the information of the object is the material of the object and that the material of the object is determined by comparing the brightness of the first and second image at corresponding points within the images.

6. A device according to claim 1, wherein the light condition is a brightness of the light.

7. A device according to claim 1, wherein the light condition is the angle of light emitted from the Vertical Cavity Surface Emitting Laser.

8. A device according to claim 1, wherein the first light condition is illuminating the object from a first direction and the second light condition is illuminating the object from a second, different, direction and the information of the object is the topography of the object, the topography being determined by photometric stereo.

9. A device according to claim 1, comprising a plurality of Vertical Cavity Surface Emitting Lasers.

10. A device according to claim 1, comprising a tip for entry into a patient, wherein the tip comprises the Vertical Cavity Surface Emitting Laser.

11. A device according to claim 1, comprising a lens arrangement to focus light onto the image sensor, wherein the Vertical Cavity Surface Emitting Laser is located adjacent to the lens arrangement.

12. A device according to claim 11 comprising a second Vertical Cavity Surface Emitting Laser located adjacent to the lens arrangement, on the side opposite the Vertical Cavity Surface Emitting Laser.

13. A device according to claim 1, comprising a structure having a reflective surface configured to change the direction of the light emitted by the Vertical Cavity Surface Emitting Laser.

14. A device according to claim 13, wherein the structure is a mirror configured to move so that the reflected light is scanned over the object.

15. A device according to claim 1, wherein the control circuitry is further configured to: annotate an image of the object using the determined object information.

16. An endoscope comprising a device according to claim 1.
